(19) United States
(12) Patent Application Publication
(10) Pub. No.: US 2026/0191965 A1
(43) Pub. Date: Jul. 9, 2026

(54) USE OF COMPOUND IN PREPARATION OF DRUG FOR TREATMENT OR PREVENTION OF MYCOBACTERIUM TUBERCULOSIS INFECTION

(71) Applicant: TenNor Therapeutics (Zhongshan) Limited, Zhongshan, Guangdong (CN)

(72) Inventors: Zhenkun MA, Suzhou, Jiangsu (CN); Ying YUAN, Suzhou, Jiangsu (CN); Shijie HE, Suzhou, Jiangsu (CN); Ling ZHANG, Suzhou, Jiangsu (CN)

(73) Assignee: TenNor Therapeutics (Zhongshan) Limited, Zhongshan, Guangdong (CN)

(21) Appl. No.: 19/132,909

(22) PCT Filed: Nov. 27, 2023
(86) PCT No.: PCT/CN2023/134307

§ 371 Date: May 27, 2025

(30) Foreign Application Priority Data
Nov. 28, 2022 (CN) 202211500665.X

(51) Int. Cl. A61K 47/54; A61P 31/06

(52) U.S. Cl. CPC A61K 47/545; A61P 31/06

(57) ABSTRACT

The present invention provides use of a rifamycin-nitroimidazole conjugate molecule, or deuterated derivatives thereof, metabolites thereof, pharmaceutically acceptable salts thereof, or prodrugs thereof in the preparation of a drug for the treatment or prevention of a disease caused by Mycobacterium tuberculosis infection. The rifamycin-nitroimidazole conjugate molecule has a structure represented by formula I.

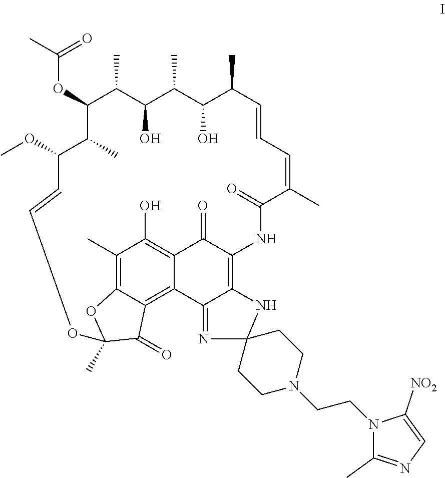 [SMILES: CO[C@H]1/C=C/O[C@@]2(C)Oc3c(C)c(O)c4c(c3C2=O)C2=NC3(CCN(CCn5c([N+](=O)[O-])cnc5C)CC3)NC2=C(NC(=O)/C(C)=C\C=C\[C@H](C)[C@H](O)[C@@H](C)[C@@H](O)[C@@H](C)[C@H](OC(C)=O)[C@@H]1C)C4=O]

The rifamycin-nitroimidazole conjugate molecule or the deuterated derivatives thereof, the metabolites thereof, the pharmaceutically acceptable salts thereof, or the prodrugs thereof in the present invention inhibit Mycobacterium tuberculosis comprising multi-drug-resistant (MDR) and extensive drug-resistant Mycobacterium tuberculosis (XDR-TB), and then are used for treating or preventing infections and a disease caused by Mycobacterium tuberculosis.

### TECHNICAL FIELD

The present disclosure relates to use of a rifamycin-nitroimidazole conjugate molecule and pertains to the technical field of pharmaceutics.

### BACKGROUND

The rifamycin-nitroimidazole conjugate molecule is a new molecular entity formed by coupling the two pharmacophores, rifamycin and nitroimidazole, via a stable covalent bond. The rifamycin-nitroimidazole conjugate molecule has a unique multi-target antibacterial mechanism. It achieves bacteriostatic and bactericidal effects through synergistic inhibition of the synthesis pathways of bacterial macromolecules such as RNA, DNA, proteins, and cell walls. The rifamycin-nitroimidazole conjugate molecule exhibited a low frequency of spontaneous resistance, a rapid bactericidal rate, a long post-antibiotic effect, and post-antibiotic sub-minimal inhibitory concentrations effect. The antibacterial properties and use of this molecule against anaerobic and microaerophilic bacteria are shown in Chinese Patent No. CN104971061B. Chinese Patent No. CN106860451A discloses its use in the inhibition of anaerobic bacteria. Chinese Patent No. CN108047250A discloses the antibacterial activity of this molecule against Nontuberculous mycobacteria. U.S. Patent No. U.S. Pat. No. 7,678,791 B2 discloses a rifamycin-nitroimidazole conjugate molecule with antibacterial activity against rifampicin-monoresistant or metronidazole-monoresistant Mycobacterium tuberculosis. Mycobacterium tuberculosis is a unique type of aerobic, Gram-positive bacteria. Its growth cycle is 20-30 times longer than that of common bacteria, imposing higher requirements on the therapeutic drugs regarding effectiveness, safety, and prevention of drug resistance. Tuberculosis (TB), caused by the bacterium Mycobacterium tuberculosis, is one of the diseases with the highest global mortality rates and currently the deadliest infectious disease. Approximately 25% of the world's population is infected with Mycobacterium tuberculosis, with 10% potentially developing active tuberculosis and 15% becoming latent tuberculosis infections. In 2016, there were 10.4 million TB cases globally, resulting in 1.7 million deaths. This included approximately 1 million pediatric TB cases, resulting in 250,000 child deaths. Among HIV patients, 40% of deaths were attributed to TB infection. In China, tuberculosis ranks second in both incidence and mortality among infectious diseases and is classified as one of the ten major infectious diseases threatening public health. Treatment for drug-sensitive tuberculosis requires a two-month intensive phase using four first-line anti-TB drugs, followed by a four-month continuation phase with two drugs. The prolonged treatment duration and poor patient compliance often result in increased drug resistance and reduced cure rates.

Mycobacterium tuberculosis has robust intrinsic resistance mechanisms (including its unique drug-impermeable cell wall structure and multiple efflux pump systems) and acquired resistance mechanisms (via spontaneous genetic mutations). Globally, the increasing incidence of tuberculosis cases caused by multiple drug resistance (MDR, resistance to isoniazid and rifampicin) and extensive drug resistance (XDR, resistance to isoniazid and rifampicin and to quinolone and one second-line anti-TB drug) poses a major threat to human health. In 2016, conservative estimates indicated 500,000 MDR-TB cases worldwide, of which 64% occurred in India, Indonesia, China, Pakistan, Nigeria, and South Africa. Among newly diagnosed TB patients, approximately 3.7% are MDR-TB cases. XDR-TB cases have been reported in 84 countries, accounting for 9% of all MDR-TB cases. The WHO recently recommended the treatment regimen for MDR-TB that requires a 4-month combination therapy with four second-line drugs followed by a 5-month therapy with two second-line drugs, with a reported treatment success rate of 85%. However, 98% of XDR-TB patients lack access to effective treatment.

Another challenge in tuberculosis treatment is that after treatment with drugs, in lung tissue granulomas or under hypoxic conditions, Mycobacterium tuberculosis transitions from a growth state with high metabolic activity to a hypometabolic slow-growth state and ultimately exists in a non-replicating persistence (NRP) state—that is, latent tuberculosis infection. In the NRP state, Mycobacterium tuberculosis is completely or partially insensitive to anti-tuberculosis drugs (e.g., isoniazid), which is the main reason for the prolonged duration of anti-tuberculosis treatment. A treatment duration of 6 months or longer significantly reduces patient adherence and greatly increases the risk of drug resistance due to interrupted or incomplete treatment (Mitchison, D. and Davies, G., 2012; Alnimr, A. M., 2015). Therefore, developing drugs that killing Mycobacterium tuberculosis in the non-replicating persisting (NRP) state or block their transition into the NRP state is crucial for shortening the treatment duration of tuberculosis.

In another aspect, among patients with latent tuberculosis infection, 2-23% of infected individuals may progress to active tuberculosis when their immunity is compromised; while latent tuberculosis infection co-infected with HIV, the rate of progression to active tuberculosis is approximately 5-10% per year. Therefore, developing drugs that killing Mycobacterium tuberculosis in the NRP state or inhibit their transition into the NRP state is critical for tuberculosis prevention.

In summary, due to the unique characteristics of Mycobacterium tuberculosis and its infection, the antibacterial properties of the compound of the present patent application (such as its activity against Helicobacter pylori and Clostridioides difficile) do not necessarily correlate with its activity against Mycobacterium tuberculosis, particularly its activity against Mycobacterium tuberculosis in a hypometabolic/slow-growth state induced by hypoxia condition. Its antibacterial activity against Mycobacterium tuberculosis cannot be extrapolated from the antibacterial activity against anaerobic and microaerophilic bacteria described in Chinese Patent No. CN104971061B. These properties of the rifamycin-nitroimidazole conjugate molecule lay the foundation for its use against Mycobacterium tuberculosis infection. Patients with pulmonary tuberculosis develop granulomas in their lungs, which progress to caseous necrosis. Mycobacterium tuberculosis growing within these granulomas is in a hypoxic state, exhibiting resistance to anti-tuberculosis drugs. Therefore, developing of drugs with activity against the hypoxic state is crucial for tuberculosis treatment, particularly for shortening the duration of treatment or preventing latent tuberculosis infection from progressing to active tuberculosis.

### SUMMARY

In view of the defects in the prior art described above, the purpose of the present disclosure is to provide use of a rifamycin-nitroimidazole conjugate molecule. The rifamycin-nitroimidazole conjugate molecule can effectively inhibit and kill the main pathogenic bacteria that cause tuberculosis and thus can be used for treating tuberculosis.

The purpose of the present disclosure is achieved through the following technical solutions: use of a rifamycin-nitroimidazole conjugate molecule or deuterated derivatives thereof, metabolites thereof, pharmaceutically acceptable salts thereof, or prodrugs thereof in the manufacture of a medicament for treating a disease caused by Mycobacterium tuberculosis infection, wherein the rifamycin-nitroimidazole conjugate molecule has a structure represented by formula I:

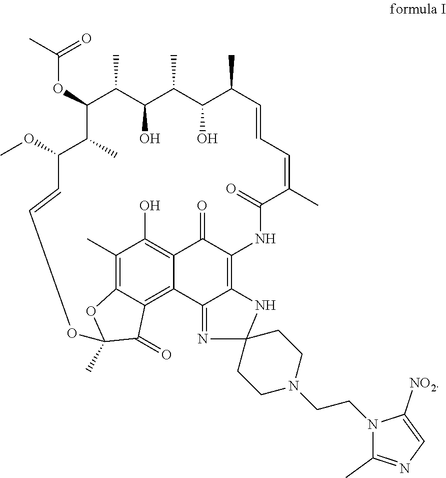 [SMILES: CO[C@H]1/C=C/O[C@@]2(C)Oc3c(C)c(O)c4c(c3C2=O)C2=NC3(CCN(CCn5c([N+](=O)[O-])cnc5C)CC3)NC2=C(NC(=O)/C(C)=C\C=C\[C@H](C)[C@H](O)[C@@H](C)[C@@H](O)[C@@H](C)[C@H](OC(C)=O)[C@@H]1C)C4=O]

In some embodiments, the Mycobacterium tuberculosis is drug-resistant Mycobacterium tuberculosis or multidrug-resistant Mycobacterium tuberculosis comprising one or more of the following drug resistance types: resistance to rifamycins, resistance to nitroimidazoles, resistance to isoniazid, resistance to pyrazinamide, resistance to macrolides, resistance to fluoroquinolones, resistance to aminoglycosides, resistance to β-lactams, resistance to tetracyclines, resistance to oxazolidinones, resistance to nitrofurans, resistance to glycopeptides, and resistance to diarylquinolines.

For example, the resistance to rifamycins may comprise: resistance to rifampicin, resistance to rifapentine, and/or resistance to rifabutin.

For example, the resistance to nitroimidazoles may comprise: resistance to metronidazole, resistance to tinidazole, resistance to ornidazole, and/or resistance to secnidazole.

For example, the resistance to macrolides may comprise: resistance to clarithromycin, resistance to azithromycin, and/or resistance to roxithromycin.

For example, the resistance to fluoroquinolones may comprise: resistance to ciprofloxacin, resistance to levofloxacin, and/or resistance to moxifloxacin.

For example, the resistance to aminoglycosides may comprise: resistance to streptomycin, resistance to amikacin, and/or resistance to ethambutol.

For example, the resistance to β-lactams may comprise resistance to ampicillin and/or resistance to amoxicillin.

For example, the resistance to tetracyclines may comprise: resistance to tetracycline, resistance to tigecycline, and/or resistance to minocycline.

For example, the resistance to oxazolidinones may comprise resistance to linezolid and/or resistance to tedizolid.

For example, the resistance to nitrofurans may comprise resistance to furazolidone.

For example, the resistance to glycopeptides may comprise resistance to vancomycin.

For example, the resistance to diarylquinolines may comprise resistance to bedaquiline and/or resistance to clofazimine.

In some embodiments, the drug resistance types do not include single resistance to rifampicin and single resistance to metronidazole.

In some embodiments, the Mycobacterium tuberculosis is Mycobacterium tuberculosis that is sensitive or drug-resistant under hypoxic conditions and/or in hypometabolic states.

In some embodiments, the Mycobacterium tuberculosis is Mycobacterium tuberculosis that, under hypoxic conditions and/or in hypometabolic states, is sensitive or comprises said drug resistance types.

The outstanding effects of the present disclosure:

The rifamycin-nitroimidazole conjugate molecule or the deuterated derivatives thereof, the metabolites thereof, the pharmaceutically acceptable salts thereof, or the prodrugs thereof of the present disclosure can effectively inhibit Mycobacterium tuberculosis and thus can be used for treating or preventing Mycobacterium tuberculosis infection, including infections with MDR/XDR strains. In addition, the rifamycin-nitroimidazole conjugate molecule or the deuterated derivatives thereof, the metabolites thereof, the pharmaceutically acceptable salts thereof, or the prodrugs thereof of the present disclosure can effectively inhibit Mycobacterium tuberculosis that is in the NRP state or is transitioning to the NRP state, thereby preventing Mycobacterium tuberculosis infection or shortening the treatment of Mycobacterium tuberculosis infection.

### BRIEF DESCRIPTION OF THE DRAWING

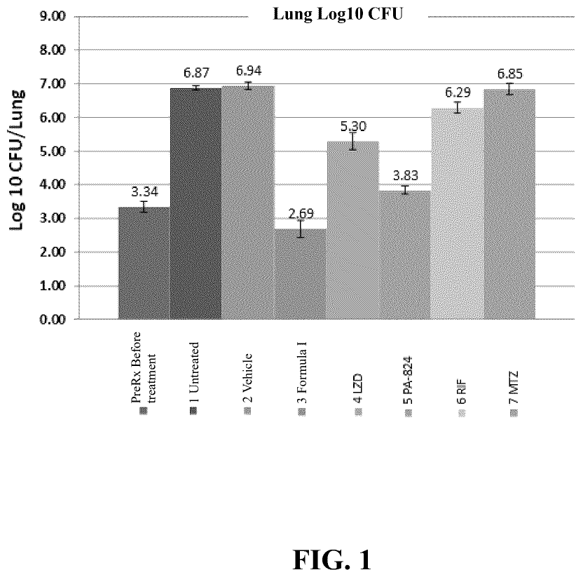

The specific features of the invention to which the present application relates are set forth in the appended claims. The features and advantages of the invention to which the present application relates may be more fully understood with reference to the exemplary embodiments and drawing described in detail below. The drawing is briefly described below: FIG. 1 is a bar graph showing the Log₁₀ CFU±SEM values in the lungs of mice infected with Erdman Mycobacterium tuberculosis (pFCA LuxAB) of Example 3 of the present disclosure.

### DETAILED DESCRIPTION

The technical solutions of the present disclosure are described in detail below to provide a clearer understanding of the technical features, purpose, and beneficial effects of the present disclosure. However, this description should not be constructed as limiting the embodiments of the present disclosure. In the following examples, the experimental methods are all conventional methods unless otherwise specified; the reagents and materials are commercially available, unless otherwise specified.

### EXAMPLES

### Example 1

This example provides use of the rifamycin-nitroimidazole conjugate molecule in combating multidrug-resistant Mycobacterium tuberculosis. In addition, its antibacterial activity in vitro against clinical multidrug-resistant Mycobacterium tuberculosis was assessed.

The drug susceptibility testing in this example was performed using the broth dilution method recommended by the Clinical and Laboratory Standards Institute (CLSI; M24-A2) guidelines. The culture medium used was a Difco™ Middlebrook 7H9 culture medium containing 10% OADC. The clinical isolates were obtained from Beijing Chest Hospital.

The control drugs were commonly used clinical anti-tuberculosis drugs: isoniazid, rifampicin, and metronidazole. The rifamycin-nitroimidazole conjugate molecule (formula I) and metronidazole were dissolved with the aid of dimethyl sulfoxide (DMSO), and rifabutin was dissolved with the aid of absolute ethanol. The other drugs were dissolved in sterile water. The concentrations of DMSO and absolute ethanol in the final test solutions did not exceed 2%.

Each clinical isolate was cultured in 7H9 culture medium in a 5% CO₂ incubator at 37° C. for 2-3 weeks until the logarithmic growth phase was reached. The clinical isolates of Mycobacterium tuberculosis were removed from the 37° C. incubator, and 200 μL of each bacterial solution was pipetted to a 96-well plate in a biosafety cabinet. In addition, 200 μL of blank 7H9 culture medium (without bacterial solution) was pipetted to the 96-well plate as a blank control. The 96-well plate was placed in a multifunctional microplate reader, and the OD values of the bacterial solutions were measured at a wavelength of 570 nm. The concentration of each strain was calculated based on an OD value of 0.1 being equivalent to 1×10⁸ CFU/mL. The strains were diluted to a concentration of 10⁶ CFU/mL. In a sterile 96-well plate, drugs and bacterial solutions were added separately. The 96-well plate was incubated in a 5% CO₂ incubator at 37° C. for 7 days, and bacterial growth was then recorded.

After 7 days, a mixture of 20 μL of 10× Alamar Blue and 50 μL of 5% Tween 80 was added to the drug-free growth control wells, followed by 24 hours of incubation at 37° C. If the color changed from blue to pink (indicating bacterial growth), the same volume of the Alamar Blue and Tween 80 mixture was added to the wells of the test drugs. After 24 hours of incubation at 37° C., the color of each well was recorded, and fluorescence values were measured at 530 nm and 590 nm using a microplate reader. The minimum inhibitory concentration (MIC) was calculated.

The results show that under the culture conditions of air containing 5% CO₂, the rifamycin-nitroimidazole conjugate molecule (formula I) exhibited similar activity to the two first-line anti-tuberculosis drugs, isoniazid and rifampicin, against sensitive tuberculosis strains, with MIC values of 0.016, 0.039, and 0.039 μg/mL, respectively. Notably, for all tuberculosis strains resistant to the drugs that constitute the molecule of formula I (rifampicin and metronidazole), the rifamycin-nitroimidazole conjugate molecule (formula I) exhibited good antibacterial activity with a MIC range of 0.5-2 μg/mL and better than isoniazid and rifampicin (Table 1). Metronidazole showed no activity against the tested strains.

TABLE 1
The antibacterial activity of the rifamycin-nitroimidazole
conjugate molecule against drug resistant Mycobacterium tuberculosis
(MIC, μg/mL)

| | Rifamycin- | | | |
|---|---|---|---|---|
| | nitroimidazole | | | |
| | conjugate | | | |
| Strain | molecule | Isoniazid | Rifampicin | Metronidazole |
| 1 | 2 | >40 | >40 | >32 |
| 2 | 0.5 | 2.5 | 5 | >32 |
| 3 | 2 | 10 | >40 | >32 |
| 4 | 2 | >40 | >40 | >32 |
| 5 | 2 | 5 | >40 | >32 |
| 6 | 1 | 0.313 | >40 | >32 |
| 7 | 2 | 5 | >40 | >32 |
| 8 | 2 | 2.5 | >40 | >32 |
| 9 | 0.5 | >40 | 20 | >32 |
| 10 | 1 | >40 | 40 | >32 |
| 11 | 0.016 | 0.039 | 0.039 | >32 |

### Example 2

This example provides use of the rifamycin-nitroimidazole conjugate molecule in combating Mycobacterium tuberculosis under hypoxic conditions.

Mycobacterium tuberculosis H37Rv (ATCC 27294) was obtained from the American Type Culture Collection (ATCC, Manassas, VA).

The rapid anaerobic dormancy (RAD) model used in this experiment was obtained with reference to the “Wayne model” (Infect. Immun. 1996, 64, 2062; Pathogens, 2018, 7, 88) and improved based on it.

Test compound concentrations: The rifamycin-nitroimidazole conjugate molecule (formula I) and the control drugs, rifampicin (RIF), rifapentine (RPT), and rifabutin (RBT), were all tested at concentrations of 5 and 20 μg/mL, and the other control drugs were tested as follows: isoniazid (INH) at 10 μg/mL and metronidazole (MET) at 20 and 50 μg/mL.

Control for anaerobic growth: Methylene blue was added to untreated control cultures. The blue dye fades away and finally disappears under anaerobe conditions. Wayne et al. correlated the fading of the dye with depletion of oxygen by direct measurement of the O₂ concentration. The graphs of his results will be applied in our experiment to determine the status of oxygen depletion. Starting precultures: Throughout the experiment, Mycobacterium tuberculosis H37Rv (wild-type), RMP-R (rifampicin-resistant strain), and RPT-R (rifapentine-resistant strain) were cultured in Dubos Tween-albumin broth. Pre-cultures were grown as 20-mL broth cultures. 2.0 mL of frozen working stock solution (H37Rv, 4.61×10⁷ CFU/mL; RMP-R, 4.29×10⁶ CFU/mL; RPT-R, 3.88×10⁶ CFU/mL) was added, and the cultures were expanded once. The cultures were incubated under aerobic conditions at 37° C. for 7 days (with vigorous stirring) to obtain exponential growing bacteria. Setting up anaerobic cultures: The culture medium was dispensed as 9-mL aliquots in 15 mm×125 mm screw cap tubes. Bacteria were added as 1 mL of a 10% suspension of OD₆₀₀=0.5-0.6 H37Rv (final dilution of bacteria culture was 1:100). Sterile rubber septa were used to ensure the anaerobic growth of the bacteria, and to be able to inject drugs under continuous anaerobe conditions. Tubes were incubated on stirring platforms at speed ‘8’ (medium stir speed to ensure adequate mixing). 30 μL of methylene blue stock solution at 500 μg/mL was added to three tubes (final concentration was 1.5 mg/L) included as controls for visual confirmation of anaerobic growth of the bacteria.

(Methylene Blue Decolorizes with Oxygen Depletion).

Plating: Bacterial cultures were serially diluted in 1:5 dilution steps in phosphate-buffered saline (PBS); dilutions 1-8 were plated onto 7H11/OADC agar. Cultures were incubated at 37° C. under normal atmosphere. Three weeks after plating the bacterial colonies were counted.

The results show that compared to the drug-free group, the rifamycin-nitroimidazole conjugate molecule (formula I) reduced the bacterial count of wild-type Mycobacterium Tuberculosis by >4 Log₁₀ CFU at both tested concentrations (20 μg/mL and 5 μg/mL) (Table 2). This indicates that the molecule of formula I exhibited a strong antibacterial activity on Mycobacterium Tuberculosis in a dormant state induced by hypoxia in vitro and was significantly superior to the individual drugs that constitute the molecule of formula I, rifabutin and metronidazole, and the other anti-tuberculosis control drugs. The study also found that metronidazole exhibited a certain inhibitory effect on the growth of both rifamycin-sensitive strains and rifamycin-resistant tuberculosis strains under anaerobic conditions. Thus, it is speculated that the synergistic activity of the two pharmacophores, rifamycin and metronidazole, may contribute to the efficient and rapid bactericidal activity of the 5 rifamycin-nitroimidazole conjugate molecule (formula I).

TABLE 2
The antibacterial activity of the rifamycin-nitroimidazole
conjugate molecule and the control drugs against
wild-type Mycobacterium Tuberculosis

| | | | | Log |
|---|---|---|---|---|
| | | Concentration | | reduction |
| Strain | Compound | (μg/mL) | CFU/mL | in CFU |
| ATCC | Rifamycin- | 20 | 2.50E+03 | 4.98 |
| 27294 | nitroimidazole | 5 | 9.75E+03 | 4.39 |
| | conjugate molecule | | | |
| | Drug-free control | NA | 2.40E+08 | 0 |
| | Isoniazid | 10 | 3.14E+08 | −0.12 |
| | Rifampicin | 20 | 1.23E+07 | 1.29 |
| | | 5 | 2.19E+07 | 1.04 |
| | Rifapentine | 20 | 1.91E+07 | 1.1 |
| | | 5 | 1.80E+07 | 1.12 |
| | Rifabutin | 20 | 7.19E+06 | 1.52 |
| | | 5 | 9.77E+07 | 0.39 |
| | Metronidazole | 50 | 1.02E+08 | 0.37 |
| | | 20 | 1.66E+08 | 0.16 |

### Example 3

This example provides the activity of the rifamycin-nitroimidazole conjugate molecule in a BALB/c acute mouse model of Mycobacterium tuberculosis infection.

Bacterial Strain. Working stocks of M. tuberculosis Erdman pFCA LuxAB were frozen in 1.5 mL aliquots and stored at −80° C. before use (pFCA LuxAB was provided by Dr. S. Franzblau, University of Illinois, Chicago). For infection, an aliquot was thawed, disrupted 20 times with a 1 ml luer-lock syringe fitted with a 26 g needle, and diluted in sterile deionized water.

The control drugs were rifampicin, PA-824, linezolid, and metronidazole. An aqueous solution of the rifamycin-nitroimidazole conjugate molecule (formula I) containing 0.5% sodium carboxymethyl cellulose (CMC-Na, w/v) and 0.5% Tween 80 (v/v) was prepared, vortexed, and ultrasonicated until the conjugate molecule was uniformly dispersed. An aqueous solution of PA-824 containing 10% 2-hydroxypropyl-β-cyclodextrin was prepared. An aqueous solution of linezolid containing 0.5% methylcellulose was prepared. Rifampicin and metronidazole were dissolved in water.

Test animals: Female 6-8 weeks old Balb/c mice were purchased from Charles River Laboratories (Wilmington, Massachusetts). The mice were rested at least one week prior to infection. Aerosol infection: Balb/c mice were infected by the aerosol route on day 0 using the Inhalation Exposure System (Glas-col Inc, Terre Haute, IN) to result in a lung bacterial load of an average of −100 colony forming units (CFU) per mouse. The M. tuberculosis Erdman pFCA LuxAB strain was used for the infection. Mice were randomized to treatment groups (6 mice per group) after aerosol infection. Three mice per infection strain were sacrificed, followed by cervical dislocation on day 1 post-aerosol infection to determine bacterial uptake. For this purpose, whole lungs were removed aseptically, homogenized in 4 ml of 1×PBS and plated without further dilution on 150×15 mm 7H11/OADC agar plates. The plates were placed in a 37° C. dry-air incubator for about 3-4 weeks before enumeration for colony formation units (CFU) and kept for about 6 weeks. Antimicrobial treatment: Treatment started day 7 after aerosol infection and was given once daily (QD) for 12 consecutive days. Compounds were given as follows: Control agents: 10 mg/kg rifampicin (RIF), 50 mg/kg PA-824, 100 mg/kg linezolid (LZD), and 200 mg/kg metronidazole (MTZ). Experimental agent: 100 mg/kg rifamycin-nitroimidazole conjugate molecule (formula I). Determination of bacterial load by CFU: In order to determine the bacterial loads at start of treatment, 6 untreated mice were humanely euthanized by CO₂ asphyxiation, followed by cervical dislocation on the day of treatment initiation (day 7) to determine the pre-treatment CFU counts in the lungs and spleens. All lung lobes and spleens were removed aseptically. The left lung lobes and spleens were homogenized in 4.5 mL of 1×PBS, and diluted serially 1:5. Dilutions 0-7 were plated on 7H11/OADC quad agar plates and incubated at 37° C. in a dry air incubator for 3-4 weeks before enumeration for CFU and kept for −5 weeks. Upper right lung lobes (superior and medial) and lower right lung lobes (inferior and post-caval) were kept at −80° C. as backup in case of contamination of the left lobe homogenates, necessitating replating.

Assessment of treatment efficacy: Three days after the final treatment (day 21), mice were humanely euthanized with CO₂ asphyxiation, followed by cervical dislocation. The left lung lobes and spleens were aseptically removed, homogenized in 4.5 mL of 1×PBS, and diluted serially 1:5. Dilutions 0-7 were plated on 7H11/OADC quad agar plates, and the plates were incubated at 37° C. in a dry air incubator for 3-4 weeks before enumeration for CFU and kept for about 5 weeks.

Determination of bacterial load by luciferase readout (RLU): In addition to determination of CFU in lungs prior to and post treatment, an additional ‘indirect’ but rapid measurement of bacterial cell numbers was taken by measuring luminescence of the luciferase expressing bacteria from lung homogenates. For this purpose, 1 mL of organ homogenates were removed and placed in 15 mL conical tubes for the luminometer assay. 2 mL of Geye's solution (8.3 g/L NH₄Cl, 1 g/L KHCO₃ in H₂O) was added to the homogenates, mixed, and the mixture was incubated for 5 minutes at room temperature to lyse red blood cells (RBCs). Then, 5 mL of PBS were then added to each of the tubes to neutralize the cell lysis solution. The homogenates were then centrifuged at 3000 RPM for 10 minutes at 4° C. The supernatants were decanted, and the homogenates were suspended in 1 mL of cold PBS. Then, 100 μL of RBC-free homogenates were mixed with 900 μL of cold PBS in luminometer tubes. Each organ sample was prepared in triplicate. The samples were run on a luminometer (Berthold AutoLumatPlus LB 953). The injector volume was 100 μL, and the injectate was 1% N-decanal in ethanol (substrate for the luciferase enzyme). The measurement time was 1 second. Ten measurements were taken per tube and added together for a cumulative relative light unit (RLU) readout per sample. The data point for each organ was calculated as the average of three RLU readouts (organ samples were prepared in triplicate) and converted to log₁₀ RLU for data analysis. Analysis of efficacy data: Lung and spleen CFU counts were log-transformed and then evaluated by a one-way ANOVA (or by Kruskal-Wallis one-way analysis of variance on ranks if data fail normality test). Differences were considered significant at the 95% level of confidence.

TABLE 3
Log₁₀ CFU counts in the lungs of mice infected with Erdman Mycobacterium tuberculosis (pFCA LuxAB)

| | | | | | | | | | | Log reduction |
|---|---|---|---|---|---|---|---|---|---|---|
| Group | Mouse | | | | | | | | | compared to |
| (mg/kg) | A | B | C | D | E | F | Average | Standard error | n* | untreated group |
| Pre-treatment control group | 3.65 | 3.67 | 3.02 | 2.88 | 3.08 | 3.76 | 3.34 | 0.16 | 6/6 | N/A |
| Group 1 untreated | 7.03 | 7.05 | 6.94 | 6.79 | # | 6.73 | 6.87 | 0.06 | 6/7 | N/A |
| Group 2 vehicle group | 6.60 | 6.84 | 6.70 | 7.00 | 7.20 | 7.29 | 6.94 | 0.11 | 6/6 | −0.07 |
| Group 3 rifamycin- | 2.48 | 2.18 | 3.02 | 2.48 | 3.80 | 2.18 | 2.69 | 0.26 | 6/6 | 4.18 |
| nitroimidazole conjugate | | | | | | | | | | |
| molecule | | | | | | | | | | |
| Group 4 linezolid | 5.86 | 4.61 | 5.76 | 4.59 | 5.13 | 5.83 | 5.30 | 0.25 | 6/6 | 1.57 |
| Group 5 PA-824 | 3.29 | 4.00 | 4.06 | 3.65 | 4.01 | 3.97 | 3.83 | 0.12 | 6/6 | 3.04 |
| Group 6 rifampicin | 6.29 | 6.55 | 6.66 | 6.24 | 5.52 | 6.46 | 6.29 | 0.17 | 6/6 | 0.58 |
| Group 7 metronidazole | 6.95 | 6.95 | 6.15 | 6.60 | 7.11 | 7.31 | 6.85 | 0.17 | 6/6 | 0.02 |

*n, the number of mice with CFU values/the number of mice at plating;
N/A = not applicable;
# mice were excluded from the study due to lack of RLU signal

Results: In this example, the activity of the rifamycin-nitroimidazole conjugate molecule (formula I) and 4 control drugs (rifampicin, PA-824, linezolid, and metronidazole) in a BALB/c acute mouse model of Mycobacterium tuberculosis infection. Treatment is initiated 7 days after a low-dose aerosol infection with M. tuberculosis Erdman pFCA LuxAB strain, and continued for 12 consecutive days. The results are shown in Table 3 and FIG. 1. In this model, the rifamycin-nitroimidazole conjugate molecule administered orally exhibited high antibacterial activity against pulmonary infection with tuberculosis bacteria in mice. The rifamycin-nitroimidazole conjugate molecule I treatment group achieved a reduction of 4 log CFU compared to the vehicle group and achieved a reduction of 0.69 log CFU compared to the pre-treatment. In contrast, the rifampicin and metronidazole monotherapy groups only achieved a reduction of 0.65 log CFU and a reduction of 0.09 log CFU, respectively, compared to the vehicle group. The activity of the rifamycin-nitroimidazole conjugate molecule was significantly superior to that of the vehicle group, the rifampicin control group, the metronidazole control group, the linezolid control group, and the PA-824 control group. Moreover, no drug-related tolerance issues were observed after 12 consecutive days of administration.

The study results show that the rifamycin-nitroimidazole conjugate molecule (formula I) of the present disclosure has in vitro (in air and under anaerobic/hypoxic conditions) and in vivo antibacterial activity against Mycobacterium tuberculosis and thus can be used for treating Mycobacterium tuberculosis infection.

In addition, the deuterated derivatives, the metabolites, the pharmaceutically acceptable salts, or the prodrugs of the rifamycin-nitroimidazole conjugate molecule described in the examples of the present disclosure can also be used for manufacturing a medicament for treating or preventing a disease caused by Mycobacterium tuberculosis infection in humans.

It should be understood that the examples and embodiments described herein are for illustrative purposes only and that various modifications or changes in light thereof will be suggested to those skilled in the art and are to be included within the spirit of the present application and the scope of the appended claims.

1. A method of treating or preventing an infection caused by Mycobacterium tuberculosis, the method comprising administering to a subject in need thereof a rifamycin-nitroimidazole conjugate molecule, a deuterated derivative thereof, a metabolite thereof, a pharmaceutically acceptable salt thereof, or a prodrug thereof, wherein the rifamycin-nitroimidazole conjugate molecule has a structure represented by formula I:

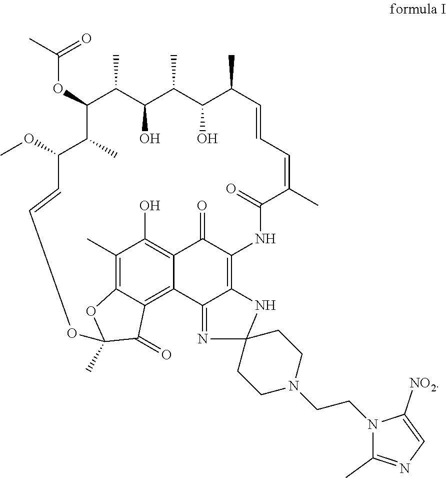 [SMILES: CO[C@H]1/C=C/O[C@@]2(C)Oc3c(C)c(O)c4c(c3C2=O)C2=NC3(CCN(CCn5c([N+](=O)[O-])cnc5C)CC3)NC2=C(NC(=O)/C(C)=C\C=C\[C@H](C)[C@H](O)[C@@H](C)[C@@H](O)[C@@H](C)[C@H](OC(C)=O)[C@@H]1C)C4=O]

2. The method according to claim 1, wherein the Mycobacterium tuberculosis is drug-resistant Mycobacterium tuberculosis or multidrug-resistant Mycobacterium tuberculosis comprising one or more of the following drug resistance types:
resistance to rifamycins, resistance to nitroimidazoles, resistance to isoniazid, resistance to pyrazinamide, resistance to macrolides, resistance to fluoroquinolones, resistance to aminoglycosides, resistance to β-lactams, resistance to tetracyclines, resistance to oxazolidinones, resistance to nitrofurans, resistance to glycopeptides, and resistance to diarylquinolines.

3. The method according to claim 2, wherein the drug resistance types do not include single resistance to rifampicin and single resistance to metronidazole.

4. The method according to claim 1, wherein the Mycobacterium tuberculosis is Mycobacterium tuberculosis that is sensitive or drug-resistant under hypoxic conditions and/or in hypometabolic states.

5. The method according to claim 2, wherein the Mycobacterium tuberculosis is Mycobacterium tuberculosis that, under hypoxic conditions and/or in hypometabolic states, is sensitive or comprises said drug resistance types.
